(19) United States
(12) Patent Application Publication
(10) Pub. No.: US 2026/0053967 A1
(43) Pub. Date: Feb. 26, 2026

(54) INTEGRATED MACHINE FOR STERILIZING AND PRESSING, ASSEMBLY LINE FOR STERILIZING AND PRESSING COMBINATION LID AND METHOD THEREFOR

(71) Applicant: Foshan Haolang Pharmaceutical Co., Ltd., Foshan (CN)

(72) Inventors: Haoran Zeng, Foshan (CN); Junhong Liu, Foshan (CN); Jie Zhao, Foshan (CN); Limin She, Foshan (CN)

(21) Appl. No.: 19/068,019

(22) Filed: Mar. 3, 2025

(30) Foreign Application Priority Data
Aug. 20, 2024 (CN) 202411141202.8

(51) Int. Cl. A61L 2/26; B29C 35/04; B29C 43/32; B29C 43/36; B29L 31/56

(52) U.S. Cl. CPC A61L 2/26; B29C 35/045; B29C 43/32; B29C 43/361; B29C 2043/3283; B29C 2043/3623; B29L 2031/565

(57) ABSTRACT

Provided in the present disclosure are an integrated machine for sterilizing and pressing, an assembly line for sterilizing and pressing a combination lid and a method therefor. The integrated machine for sterilizing and pressing includes pressing devices and a sterilization case including an outer cylinder and an inner cylinder provided in the outer cylinder. A material mold is installed in the inner cylinder and provided with a plurality of material placing holes. Each of the pressing devices includes a driving member and a pressing plate, and the driving member drives the pressing plate to move. The material mold is divided into X*Y pressing areas, an area of each pressing area is equal to an area of the pressing plate, number of the pressing devices is X*Y, each pressing device corresponds to each pressing area, and a plurality of the material placing holes are distributed on the X*Y pressing areas.

### CROSS-REFERENCE TO RELEATED APPLICATIONS

The present disclosure claims the priority of Chinese Patent Application No. 2024111412028 filed on Aug. 20, 2024 before CNIPA. All the above are hereby incorporated by reference in their entirety.

### TECHNICAL FIELD

The present disclosure relates to the technical field of sterilizing equipment, and in particular to an integrated machine for sterilizing and pressing, an assembly line for sterilizing and pressing a combination lid and a method therefor.

### BACKGROUND

A product that needs to be sterilized, then assembled and pressed, for example, a combination lid for a mouth of a filtered infusion bottle/infusion bag, has a characteristic that they need to be sterilized first and then assembled and pressed. Since a cavity is provided inside the combination lid, the cavity inside the combination lid will not be able to be fully sterilized if the combination lid is assembled and then sterilized. However, if components of the combination lid are sterilized by a traditional sterilizer and then the sterilized components are taken out and assembled, secondary contamination of the product may be caused after the components are taken out.

### SUMMARY

In order to overcome at least one defects described above in the prior art, provided in the present disclosure are an integrated machine for sterilizing and pressing, an assembly line for sterilizing and pressing a combination lid and a method therefor, which can solve a problem that a component structure provided with an enclosed cavity cannot achieve a full sterilization effect.

Technical solutions adopted by the present disclosure to solve a problem of the present disclosure are as follows:
An integrated machine for sterilizing and pressing includes a sterilization case and pressing devices.

The sterilization case includes an outer cylinder and an inner cylinder. The inner cylinder is provided in the outer cylinder, a material mold is installed in the inner cylinder, and the material mold is provided with a plurality of material placing holes.

Each of the pressing devices includes a driving member and a pressing plate, and the driving member drives the pressing plate to move.

The material mold is divided into X*Y pressing areas, an area of each of the pressing areas is equal to an area of the pressing plate, the number of the pressing devices is X*Y, each of the pressing devices corresponds to each of the pressing areas, and a plurality of the material placing holes are distributed on the X*Y pressing areas.

By adopting the above solution, the material mold and pressing devices are provided in the sterilization case, which can complete the pressing directly in the sterilization case after the sterilization of the components to be sterilized has been completed, thus realizing the full sterilization of the components to be sterilized without causing secondary contamination.

In addition, since a connection strength between the inner cylinder and the outer cylinder is relatively low due to structural characteristics of the inner cylinder of a porous type, by dividing the material mold into X*Y pressing areas and providing the pressing devices according to the number and position of the pressing areas, and by adopting a block pressing method, it can be ensured that the connection between the inner cylinder and the outer cylinder is not damaged.

In some implementations, a fixing frame is provided in the inner cylinder, N layers of the material mold and N layers of the pressing devices are provided on the fixing frame along a height direction of the inner cylinder, and each of the layers of the material mold corresponds to each of the layers of the pressing devices.

By adopting the above solution, a space of the inner cylinder can be fully utilized and the production efficiency per unit volume can be improved by providing the plurality of layers of material mold and the plurality of layers of pressing devices along the height direction of the inner cylinder.

In some implementations, the pressing plate is connected with a guiding structure, and a guiding direction of the guiding structure is parallel to a direction in which the driving member drives the pressing plate to move.

By adopting the above solution, the component structures are not fixedly placed on the material mold and any offset in the movement of the pressing plate may result in the destruction of the component structures in the entire pressing areas, so that the setting of the guiding structure ensures that the pressing plate moves in a straight line in a predetermined direction, thereby avoiding being offset or tilted in the entire pressing process, and ensuring the precision and consistency of the pressing.

In some implementations, the driving member includes a servo electric cylinder, the servo electric cylinder is provided with a telescopic rod, and the telescopic rod is fixedly connected to the pressing plate so as to drive the pressing plate to reciprocate with the telescopic rod.

By adopting the above solution, since the servo electric cylinder is capable of providing high-precision position control and maintaining a high degree of accuracy even in the environment of high temperature, high pressure, and high humidity, a consistent effect can be achieved for each pressing, and more importantly, due to the relatively low connection strength between the inner cylinder and the outer cylinder, a requirement for torque stability is extremely high. In addition, the servo electric cylinder is capable of adapting to a harsh environment, and factors such as waterproof, dustproof, and high temperature resistance are generally considered in a design of the servo electric cylinder, so that the servo electric cylinder can work continuously and stably in the environment of high temperature, high pressure, and high humidity.

In some implementations, the guiding structure includes guiding rods and a guiding plate, an avoidance hole is provided at a central part of the guiding plate, the telescopic rod passes through the avoidance hole and is fixedly connected to the pressing plate, the guiding plate is provided with a plurality of guiding holes along a circumferential direction of the servo electric cylinder, an end of each of the guiding rods passes through or is inserted into the corresponding guiding hole and is slidable relative to the corresponding guiding hole, another end of each of the guiding rods is fixedly connected to the pressing plate, and both a number and a position of the guiding rods correspond to those of the guiding holes.

The specific guiding rods cooperating with the guiding holes can ensure that the pressing plate moves in a straight line in the predetermined direction, thereby avoiding being offset or tilted in the pressing process, and ensuring the precision and consistency of the pressing. The setting of the plurality of guiding holes and the plurality of guiding rods can effectively reduce the swaying of the pressing plate during movement, thereby improving the stability of the entire system.

In some implementations, the material mold is provided with a bending-resistant reinforcing structure.

By adopting the above solution, due to a need to ensure a sterilization effect in the sterilization case, it is not appropriate that a relatively large quantity of support structures and mounting structures are provided in the sterilization case, the fixing frame can only support the edges of three sides of the material mold, and the central part of the material molds produces a relatively large deformation in the pressing process, which results in an inclination of the component structures on the basis of such large deformation, thereby resulting in the destruction of the component structures in the entire pressing areas.

In some implementations, the bending-resistant reinforcing structure is a stress sandwich layer, the material mold includes an upper structural plate and a lower structural plate, and the upper structural plate, the stress sandwich layer, and the lower structural plate are fixedly connected in sequence.

By adopting the above solution, the stress sandwich layer can significantly improve the bending resistance of the material mold, and especially in the environment of high temperature, high pressure and high humidity, the stress sandwich layer can effectively prevent the material mold from undergoing a large bending deformation due to the external press force. The stress sandwich layer enhances the overall structural stability of the material mold and ensures that the material mold is not easily deformed in the pressing process, thereby ensuring the quality and consistency of the product.

More importantly, by using the stress sandwich layer, a thickness of the material mold can be reduced without sacrificing strength, so as to reduce self-weight of the material mold, and reduce load of the connection between the inner cylinder and the outer cylinder, thereby greatly reducing a possibility of the connection structure of the inner cylinder and the outer cylinder being damaged. Moreover, the stress sandwich layer can effectively disperse an external force, reduce stress concentration in key parts of the material mold, and prolong a service life of the material mold.

In some implementations, the stress sandwich layer is divided into two stress areas from a center of the stress sandwich layer to edges of the stress sandwich layer, one of the two stress areas at a central part of the stress sandwich layer is a first stress area, the other of the two stress areas at an edge part of the stress sandwich layer is a second stress area, and stress on the first stress area is greater than stress on the second stress area.

By adopting the above solution, the stress sandwich is divided into different stress areas, which can optimize the stress distribution of the entire material mold, so that the stress can be dispersed more uniformly when the material mold subjected to force and the relatively large deformation of the material mold caused by the excessively high local stress is reduced, thereby avoiding resulting in an excessive angle inclination during pressing the components.

Further provided in the present disclosure is an assembly line for sterilizing and pressing a combination lid, including the above integrated machine for sterilizing and pressing, which is configured to sterilize and press the combination lid. The combination lid includes at least two components, two of the at least two components are correspondingly placed in one of the material placing holes, and the pressing devices press the two components together and then an enclosed cavity is formed between the two components.

By adopting the above solution, the assembly line for sterilizing and pressing integrates the sterilization and pressing operations, which simplifies the production process and improves the production efficiency, and by integrating the sterilization and pressing operations in the same space, it can be ensured that the components of the combination lid are fully sterilized and secondary contamination is not occurs on the sterilized components of the combination lid, thereby improving the cleanliness and quality consistency of the product.

Further provided in the present disclosure is a method for sterilizing and pressing a combination lid, using the above integrated machine for sterilizing and pressing. The method specifically includes following steps:
S1, stacking two components forming a combination lid in each of the material placing holes;
S2, placing the material mold into the integrated machine for sterilizing and pressing and closing the door of the integrated machine for sterilizing and pressing;
S3, vacuuming the integrated machine for sterilizing and pressing to a vacuum state and heating up inside the integrated machine for sterilizing and pressing;
S4, spraying sterilizing gas into a sterilizing system inside the integrated machine for sterilizing and pressing, then setting pressure inside the integrated machine for sterilizing and pressing to a range of 0.1 MPa to 0.3 MPa and temperature inside the integrated machine for sterilizing and pressing to 115° C. to 121° C., and continuously spraying sterilizing gas for 15 minutes to 30 minutes to perform sterilization;
S5, vacuuming the integrated machine for sterilizing and pressing to the vacuum state again;
S6, drying inside the integrated machine for sterilizing and pressing to obtain a sterilized combination lid;
S7, pressing down X*Y pressing devices one by one until the last pressing device completes the pressing action, so as to complete pressing of the combination lid; and
S8, adjusting air pressure inside the integrated machine for sterilizing and pressing and opening a case door of the integrated machine for sterilizing and pressing, so as to complete production of the combination lid.

By adopting the above solution, the production process is simplified and the sterilization effect of the production of the combination lid is ensured by integrating the sterilization and pressing operations. In addition, a material handling time and labor cost are also reduced, thus improving the production efficiency. The sterilization and pressing operations are closely combined, ensuring that the combination lid is pressed immediately after sterilization, reducing the risk of cross-contamination and improving hygiene standards and quality consistency. Two vacuums help to improve the sterilization effect and ensure that the combination lid is completely cleared of micro-organisms during the sterilization. The removal of sterilized gas residues by the second vacuum improves the efficiency of the drying process and ensures that the lid reaches an ideal drying state before pressing.

In summary, the integrated machine for sterilizing and pressing, the assembly line for sterilizing and pressing the combination lid and the method therefor provided in the present disclosure have the following technical effects:
1. A material mold and a pressing devices are provided in the sterilization chamber, so that the pressing can be completed directly in the sterilization case after the sterilization of the components to be sterilized has been completed, thus realizing the full sterilization of the components to be sterilized.
2. By directly completing the pressing inside the sterilization case, the possibility of re-exposure of the combination lid to the external environment after sterilization is avoided, thereby reducing the risk of secondary contamination.
3. Since a connection strength between the inner cylinder and the outer cylinder is relatively low due to structural characteristics of the inner cylinder of a porous type, by dividing the material mold 13 into X*Y pressing areas and providing the pressing devices according to the number and position of the pressing areas, and by adopting a block pressing method, it can be ensured that the connection between the inner cylinder and the outer cylinder is not damaged.

### BRIEF DESCRIPTION OF THE DRAWINGS

FIG. 1 is a schematic structural diagram in a side view of an integrated machine for sterilizing and pressing in the present disclosure;

FIG. 2 is a schematic structural diagram in a front view of an integrated machine for sterilizing and pressing in the present disclosure;

FIG. 3 is a schematic structural diagram of a material mold in the present disclosure;

FIG. 4 is a schematic structural diagram of a driving member and a pressing plate in the present disclosure;

FIG. 5 is a schematic diagram illustrating stress area division of a stress sandwich layer in the present disclosure; and

FIG. 6 is a schematic structural diagram in a sectional view of the material mold in the present disclosure.

The meanings of the accompanying drawing marks are as follows: 11 outer cylinder, 12 inner cylinder, 13 material mold, 131 stress sandwich layer, 132 upper structural plate, 133 lower structural plate, 141 pressing area, 142 first stress area, 143 second stress area, 15 material placing hole, 16 fixing frame, 21 driving member, 211 servo electric cylinder, 212 telescopic rod, 22 pressing plate, 23 guiding structure 231 guiding rod, 24 guiding plate.

### DETAILED DESCRIPTION

In order to facilitate the understanding of the embodiments of the present disclosure, specific embodiments in conjunction with the attached drawings are further explained hereinafter, and each embodiment does not constitute a limitation to the embodiments of the present disclosure.

In the description of the present disclosure, it is to be noted that the terms “up”, “down”, “front”, “back”, “left”, “right”, “vertical”, “horizontal”, “top”, “bottom”, “inside”, “outside” and other orientation or position relationships are based on the orientation or position relationships shown in the attached drawings. It is only intended to facilitate description of the present disclosure and simplify description, but not to indicate or imply that the referred device or element has a specific orientation, or is constructed and operated in a specific orientation. Therefore, they should not be construed as a limitation of the present disclosure.

Unless otherwise defined, all terms including technical and scientific terms used herein have the same meaning as commonly understood by those skilled in the art to which the present disclosure belongs. The terms used herein in the specification of the present disclosure are used only to describe specific embodiments and are not intended as a limitation of the disclosure.

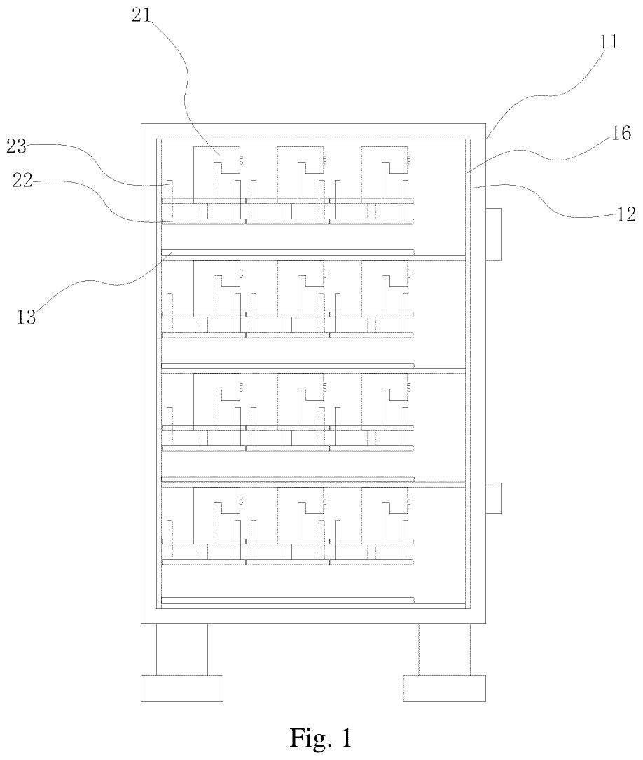

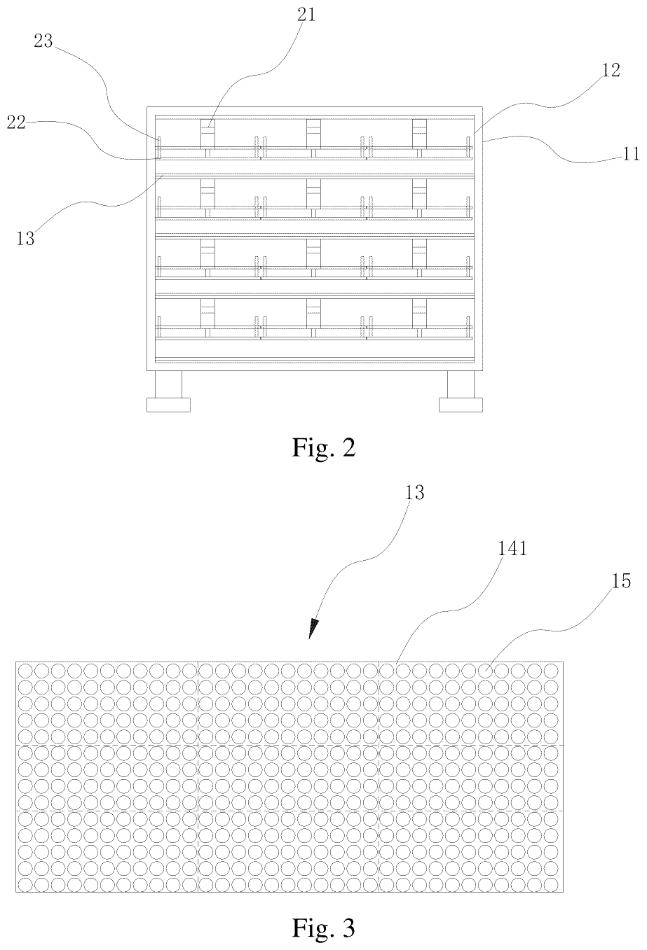

Referring to FIGS. 1 to 3, disclosed in the present disclosure is an integrated machine for sterilizing and pressing. The integrated machine for sterilizing and pressing includes a sterilization case and pressing devices. The sterilization case includes an outer cylinder 11 and an inner cylinder 12. The inner cylinder 12 is provided in the outer cylinder 11. A material mold 13 is installed in the inner cylinder 12, and the material mold 13 is provided with a plurality of material placing holes 15. Each of the pressing devices includes a driving member 21 and a pressing plate 22, and the driving member 21 drives the pressing plate 22 to move. The material mold 13 is divided into X*Y pressing areas 141, an area of each of the pressing areas 141 is equal to an area of the pressing plate 22, number of the pressing devices is X*Y, each of the pressing devices corresponds to each of the pressing areas 141, and a plurality of the material placing holes 15 are distributed on the X*Y pressing areas 141.

Specifically, the sterilization case includes an outer cylinder 11 and an inner cylinder 12, the inner cylinder 12 is configured to be installed with the material mold 13, and the material mold 13 is provided with a plurality of material placing holes 15. In an operation of sterilizing and pressing component structures (hereinafter referred to as combination lids) provided with an enclosed cavity, components composing the combination lids are stacked and placed in the material placing holes 15, and the components of each combination lid are stacked and placed in one of the material placing holes 15. The pressing device includes a driving member 21 and a pressing plate 22. The driving member 21 is capable of driving the pressing plate 22 to achieve a telescopic function, and may specifically be a pneumatic, a hydraulic telescopic rod 212, etc., which is not limited herein. The pressing plate 22, by using a conventional rigid plate, may not produce a significant deformation, and can work normally in a state of high temperature, high pressure, and high humidity inside the sterilization case. The material mold 13 is divided into X*Y pressing areas 141, i.e., a large surface of the material mold 13 is divided into X rows and Y columns of pressing areas 141. X*Y pressing devices are provided, the number, position and size of which correspond to the number, position and size of the X rows and Y columns of pressing areas 141. Specifically, since a connection strength between the inner cylinder 12 and the outer cylinder 11 is relatively low due to structural characteristics of the inner cylinder 12 of a porous type, by dividing the material mold 13 into X*Y pressing areas 141 and providing the pressing devices according to the number and position of the pressing areas 141, and by adopting a block pressing method, it can be ensured that the connection between the inner cylinder 12 and the outer cylinder 11 is not damaged. In a technical solution that is easy to be thought of, one pressing device is driven to move by a mechanical arm or a three-axis driving device so as to realize a blocking effect on a plurality of pressing areas 141. In the technical solution of using the single pressing device for pressing, in addition to the driving member 21 such as the mechanical arm or the three-axis driving device, a device that facilitates the positioning of the pressing device, for example, a grating, visual recognition probe, etc., is also required to be installed due to the complex environment of high temperature, high pressure and high humidity inside the sterilization case. However, the grating in the environment of high temperature, high pressure and high humidity may produce an error, and then in a case of the material placing holes 15 being densely provided, the error in the process of prolonged use may lead to a cliff decline in a yield rate. In addition, in combination with the use of sterilizing gases in the sterilizing process, the grating and the visual recognition probe may further produce an error that can reduce the yield rate.

In addition, due to the limited space in the sterilization case, in order to improve the production efficiency, the material placing holes 15 are provided in the material mold 13 as many as possible, i.e., the number of the material placing holes 15 arranged in the material mold 13 is increased as much as possible. On the basis of a structure of the material placing holes 15 being provided in the material mold 13 densely, the strength of the material mold 13 itself is reduced, thereby the use of block pressing can address the impact of the material mold 13 correspondingly.

In the present embodiment, in order to improve the production efficiency of the combination lid, a fixing frame 16 is provided inside the inner cylinder 12, N layers of material mold 13 and N layers of pressing devices are provided on the fixing frame 16 along a height direction of the inner cylinder 12, and each of layers of the material mold 13 corresponds to each of layers of the pressing devices. Therefore, space of the inner cylinder 12 can be fully utilized, and production efficiency in a unit volume can be improved.

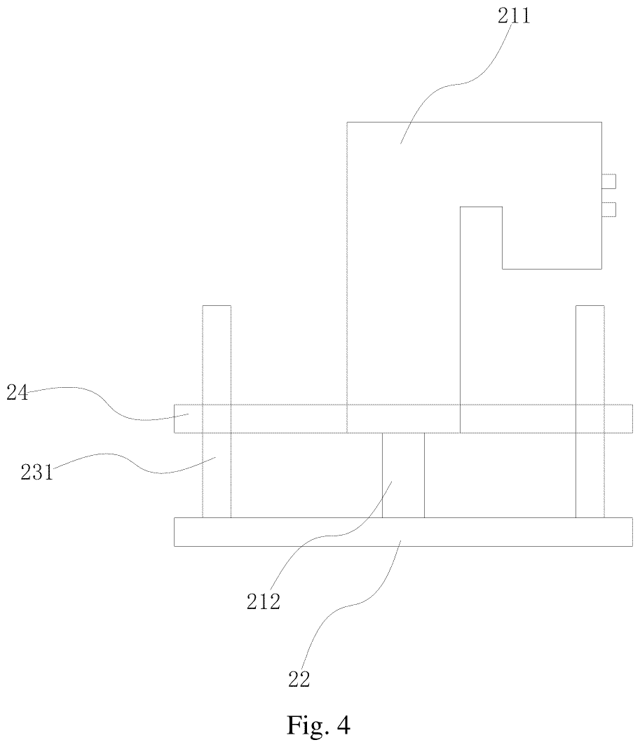

Referring to FIGS. 1 to 4, in some embodiments, in order to ensure the accuracy of the movement of the pressing plate 22, the pressing plate 22 is connected with a guiding structure 23, and a guiding direction of the guiding structure 23 is parallel to a direction in which the driving member 21 drives the pressing plate 22 to move. Specifically, since the component structures are not fixedly placed on the material mold 13, i.e., the components composing the combination lid are naturally stacked together, and any offset in the movement of the pressing plate 22 results in the destruction of the component structures in the entire pressing areas 141, so that the setting of the guiding structure 23 ensures that the pressing plate 22 moves along a straight line in a predetermined direction, thereby avoiding being offset or tilted in the entire pressing process, and ensuring the precision and consistency of the pressing.

In some embodiments, the driving member 21 includes a servo electric cylinder 211, the servo electric cylinder 211 is provided with a telescopic rod 212, and the telescopic rod 212 is fixedly connected to the pressing plate 22 to drive the pressing plate 22 to reciprocate with the telescopic rod 212. Specifically, since the servo electric cylinder 211 is capable of providing high-precision position control and maintaining a high degree of accuracy even in the environment of high temperature, high pressure, and high humidity, a consistent effect can be achieved for each pressing, and more importantly, due to the relatively low connection strength between the inner cylinder 12 and the outer cylinder 11, a requirement for torque stability is extremely high. In addition, the servo electric cylinder 211 is capable of adapting to a harsh environment, and factors such as waterproof, dustproof, and high temperature resistance are generally considered in a design of the servo electric cylinder 211, so that the servo electric cylinder 211 can work continuously and stably in the environment of high temperature, high pressure, and high humidity.

On the basis of the structure of the guiding structure 23, the guiding structure 23 includes guiding rods 231 and a guiding plate 24, an avoidance hole is provided on a central part of the guiding plate 24, the telescopic rod 212 passes through the avoidance hole and is fixedly connected to the pressing plate 22, the guiding plate 24 is provided with a plurality of guiding holes in a circumferential direction of the servo electric cylinder 211, an end of each of the guiding rods 231 passes through or is inserted into the corresponding guiding hole and is slidable relative to the corresponding guiding hole, another end of each of the guiding rods 231 is fixedly connected to the pressing plate 22, and both the number and position of the guiding rods 231 are provided in correspondence with those of the guide holes. Specifically, the guiding rods 231 cooperating with the guiding holes can ensure that the pressing plate 22 moves in a straight line in the predetermined direction, thereby avoiding being offset or tilted in the pressing process, and ensuring the precision and consistency of the pressing. The setting of the plurality of guiding holes and the plurality of guiding rods 231 can effectively reduce the swaying of the pressing plate 22 during movement, thereby improving the stability of the entire system.

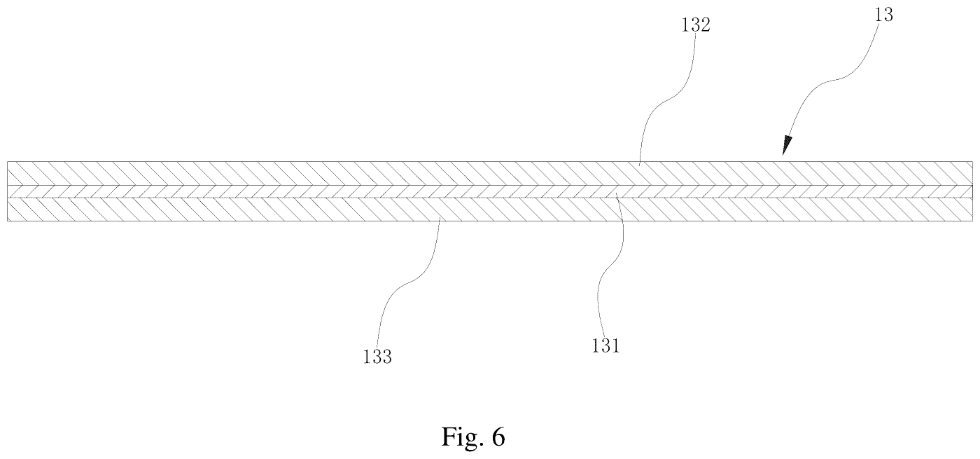

Referring to FIGS. 3, 5, and 6, in some embodiments, the material mold 13 is provided with a bending-resistant reinforcing structure in order to improve the bending-resistant performance of the material mold 13. Specifically, due to a need to ensure a sterilization effect in the sterilization case, it is not appropriate that a relatively large quantity of support structures and mounting structures are provided in the sterilization case, and the fixing frame 16 can only support the edges of three sides of the material mold 13. In combination with the fact that the capacity of the inner cylinder 12 of the small sterilization case on the present market at present is in a range of 0.5 m³ to 10 m³, a length of a single side of the material mold 13 may be in a range of 1 m to 2 m if the above structure with the support effect on the material mold 13 is adopted. On the basis of the long span, and superimposed the structural basis of relatively low connection structure between the inner cylinder 12 and the outer cylinder 11, a relatively high weight of the material mold 13 itself can be caused if a high-strength stainless steel material or another high-strength bending material is used. In order to ensure that connection structure between the inner cylinder 12 and the outer cylinder 11 is not damaged, a pressure that the corresponding pressing device is allowed to exert on the combination lid may be in a relatively small range, which results in a reduction in types of the combination lids that can be pressed together by the integrated machine for sterilizing and pressing in the present disclosure. In addition, the sterilization of many equipment in medical field needs to rely on intervention of substances such as acids, alkalis, strong oxidants, halogens and other substances, in combination with an environment of high temperature and high humidity in the sterilization case, materials such as stainless steel are highly susceptible to corrosion, resulting in a decline in a service life of the material molds 13. On the basis of the above limitations, if a high-strength material such as stainless steel is used, an application scope of the integrated machine for sterilizing and pressing of the present disclosure is also limited.

Therefore, the material mold 13 in the present embodiment can be made of a polymer material that can withstand an environment of high temperature, high pressure and high humidity. Certainly, the material mold 13 also needs to have a certain bending resistance performance. The bending-resistant reinforcing structure may be a reinforcing rib, and is provided on a rear surface of the material mold 13 in a form of the reinforcing rib, so that the bending resistance of the material mold 13 is improved.

Referring to FIGS. 3, 5, and 6, in other embodiments, the bending-resistant reinforcing structure is a stress sandwich layer 131. The material mold 13 includes an upper structural plate 132 and a lower structural plate 133, and the upper structural plate 132, the stress sandwich layer 131, and the lower structural plate 133 are fixedly connected in sequence.

Specifically, in a processing process, the stress sandwich layer 131 is tensioned in advance, and then the upper structural plate 132 and the lower structural plate 133 are glued and fixed with the stress sandwich layer 131. Certainly, an anchoring manner may also be used to carry out the fixed connection of the upper structural plate 132, the stress sandwich layer 131, and the lower structural plate 133. The stress sandwich layer 131 may be tensile by using a carbon fiber-reinforcing composite material, which is capable of adapt to the environment of high temperature, high pressure, and high humidity in the sterilization chamber. The stress sandwich layer 131 in the above structure can significantly improve the bending resistance of the material mold 13, and especially in the environment of high temperature, high pressure and high humidity, the stress sandwich layer 131 in the above structure can effectively prevent the material mold 13 from undergoing a large bending deformation due to the indirect press force applied by the pressing device, i.e., the pressing plate 22 exerts press force on the combination lids and the combination lids exert press force on the material mold 13. The stress sandwich layer 131 enhances the overall structural stability of the material mold 13 and ensures that the material mold is not easily deformed in the pressing process, thereby ensuring the quality and consistency of the product.

More importantly, by using the stress sandwich layer 131, a thickness of the material mold 13 can be reduced without sacrificing strength, so as to reduce self-weight of the material mold 13, and reduce load of the connection between the inner cylinder 12 and the outer cylinder 11, thereby greatly reducing a possibility of the connection structure of the inner cylinder 12 and the outer cylinder 11 being damaged. In contrast, an upper limit of the press force that can be applied by the pressing device in the pressing process is improved. Moreover, the stress sandwich layer 131 can effectively disperse an external force, reduce stress concentration in key parts of the material mold 13, and prolong a service life of the material mold.

In addition, in a case of the material with strong bending resistance being used to make the material mold 13, it is assumed that the components of the combination lid are combined by using the snap structure for the sake of convenience. In a pressing process of the pressing device, a force may be transmitted to the material mold 13 through the combination lid, a stress curve to which the material mold 13 is subjected may be gradually increased, and when deformation of the snap reaches a maximum value, a press force absorbed by the deformation of the components of the combination lids reach a maximum value. Then a press force of the pressing device may all be indirectly displayed in the stress monitoring data of the material mold 13, and a slope of an analytical graph of the stress on the material mold 13 is greater, i.e., the stress increases faster in unit time, which corresponds to a scenario in which the material mold 13 needs to be able to withstand a higher rate of increase of stress, and has higher requirements for the strength of the material mold 13.

However, for the structure in which the upper structural plate 132, the stress sandwich layer 131, and the lower structural plate 133 are fixedly connected in sequence, the upper structural plate 132 and the lower structural plate 133 may be made of a material whose bending resistance strength is lower than that in the above case. Specifically, it is assumed that the components of the combination lid are combined by using the snap structure for the sake of convenience. In the pressing process of the pressing device, a force may be transmitted to the material mold 13 through the combination lid, a stress curve to which the material mold 13 is subjected may be gradually increased. When the stress is within a range in which the snap structure is capable of producing elastic deformation, and in which the upper structural plate 132 and the lower structural plate 133 both are capable of producing a downwardly bending elastic deformation, the stress as well as the corresponding deformation to which the snap is subjected, and, the stress as well as the corresponding deformation to which the upper structural plate 132 and the lower structural plate 133 are subjected may increase alternately. When deformation of the snap reaches a maximum value, the press force absorbed by the deformation of the components of the combination lids reach a maximum value, the upper structural plate 132 and the lower structural plate 133 may still have a corresponding deformation space, so that the stress curve of the upper structural plate 132 and the lower structural plate 133 is more smooth, i.e., the stress curve of the material mold 13 is more smooth, thereby reducing the impact force of the pressing device on the material mold 13, and reducing a requirement for strength of the connection structure between the inner cylinder 12 and the outer cylinder 11. Compared to a conventional thinking of increasing the strength of the material mold 13 by using a material mold 13 with relatively high strength so as to supply sufficient support and bending resistance, in the present disclosure, the appropriate elastic deformation is used to moderate a change rate of the stress curve to which the material mold 13 is subjected, so as to expand the application scope of the integrated machine for sterilizing and pressing.

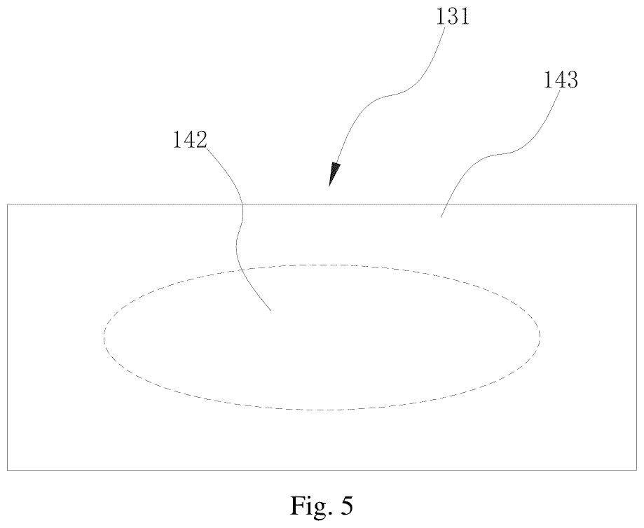

As shown in FIG. 5, on a basis of a structure in which the bending-resistant reinforcing structure is the stress sandwich layer 131, the stress sandwich layer 131 is divided into two stress areas from a center of the stress sandwich layer 131 to edges of the stress sandwich layer 131, one of the two stress areas at a central part of the stress sandwich layer 131 is a first stress area 142, the other of the two stress areas at an edge part of the stress sandwich layer 131 is a second stress area 143, and stress on the first stress area 142 is greater than stress on the second stress area 143.

A specific production process may be as follows: a tensile force of a tensioning equipment for pre-tensioning the stress sandwich layer 131 is adjusted to N1, and the upper structural plate 132 and the lower structural plate 133 are coated with glue on a plate surface corresponding to the first stress area 142, and then pasted at the stress sandwich layer 131; after the gluing is completed, the tensile force of the tensioning equipment is lowered to N2, and gluing is performed in a gap between the upper structural plate 132, the stress sandwich layer 131, and the lower structural plate 133; and after the second stress area 143 has been glued, the tension of the tensioning equipment on the stress sandwich layer 131 is released, and the excess glue and the excess stress sandwich layer 131 between the upper structural plate 132 and the lower structural plate 133 are cut off, so as to obtain the material mold 13 with two stress areas.

It should be noted that since both the upper structural plate 132 and the lower structural plate 133 are provided with through holes constituting the material placing holes 15, and the material placing holes 15 are provided with a relatively large density, it is still possible to achieve good gluing of the second stress area 143 after the first stress area 142 has been glued. Certainly, a structure such as a glue guiding groove may also be provided as needed to improve the gluing effect, which is not limited herein.

In addition, it should be noted that in order to better show the demarcation edge of the first stress area 142 and the second stress area 143, the treatment of hiding the material placing holes 15 are processed in FIG. 5, and in an actual embodiment, the stress sandwich layer 131 may be provided with through holes corresponding to the material placing holes 15.

When the upper structural plate 132 and the lower structural plate 133 are made of polyether ether ketone (PEEK), a ratio of N1 to N2 is 1:0.93. In this way, it may be ensured that, in a case in which the upper structural plate 132 and the lower structural plate 133 are bent upward by the stress sandwich layer 131 in a conventional state, a tilting angle of the combination lids in the material placing holes 15 is not greater than 3 degrees, so as to ensure that in the pressing process of the combination lids, the combination lids are not damaged, thereby ensuring the yield rate.

Further provided in the present disclosure is an assembly line for sterilizing and pressing a combination lid, including the above integrated machine for sterilizing and includes at least two components. Two of the at least two components are correspondingly placed in one of the material placing holes 15. The pressing devices press the two components together, and then an enclosed cavity is formed between the two components.

Specifically, in the assembly line for sterilizing and pressing the combination lid, the above integrated machine for sterilizing and pressing is used to complete the sterilization and pressing process, and by integrating the sterilization and pressing operations in a same space, it can be ensured that the components of the combination lid are fully sterilized and secondary contamination is not occurs on the sterilized components of the combination lid, thereby improving the cleanliness and quality consistency of the product.

Further provided in the present disclosure is a method for sterilizing and pressing a combination lid, using the above integrated machine for sterilizing and pressing. The method specifically includes following steps:
S1, two components composing a combination lid are stacked in each of the material placing holes 15, so as to prepare for subsequent sterilizing and pressing operations.
S2, the material mold 13 is placed into the integrated machine for sterilizing and pressing and a case door of the integrated machine for sterilizing and pressing is closed, so as to create an enclosed environment for the sterilization process.
S3, the integrated machine for sterilizing and pressing is vacuumed to a vacuum state and heat up. Air inside the integrated machine for sterilizing and pressing is removed by vacuuming to reduce effects of oxygen and moisture, and then heat up, so as to create a necessary condition for a subsequent sterilization process.
S4, sterilizing gas is sprayed into a sterilizing system inside the integrated machine for sterilizing and pressing, then pressure inside the integrated machine for sterilizing and pressing is set to a range of 0.1 MPa to 0.3 MPa and temperature inside the integrated machine for sterilizing and pressing is set to a range of 115° C. to 121° C., and the sterilizing gas is continuously sprayed for 15 minutes to 30 minutes to perform sterilization. The sterilizing gas is sprayed at the set pressure and temperature to perform sterilization, so as to kill or eliminate the microorganisms on the combination lid and ensure the hygienic safety of the product.
S5, the integrated machine for sterilizing and pressing is vacuumed to a vacuum state again. After the sterilization process is finished, the vacuuming is performed again to remove the residual sterilized gas, so as to prepare for a subsequent drying process.
S6, the inside of the integrated machine for sterilizing and pressing is dried to obtain a sterilized combination lid. The moisture in the combination lid is removed by drying, so as to ensure that the combination lid reaches a desirable dry state before being pressing, thereby improving a pressing effect.
S7, X*Y pressing devices are pressed down one by one until the last pressing device completes the pressing action, so as to complete pressing of the combination lid. The pressing devices are pressed down one by one, so that the two components are pressed together to form an enclosed cavity, thereby completing the assembly of the combination cover, and improving the stability of the pressing process of the integrated machine for sterilizing and pressing, which has been described above in detail, and is not repeated herein.
S8, air pressure inside the integrated machine for sterilizing and pressing is adjusted and a case door of the integrated machine for sterilizing and pressing is opened, so as to complete production of the combination lid.

The technical means disclosed in the solution of the present disclosure are not limited to those disclosed in the embodiments mentioned above but also include technical solutions consisting of any combination of the above technical features. It should be noted that for those skilled in the art, a plurality of improvements and modifications may be made without departing from the principles of the present disclosure, which should also be considered as the scope of protection of the present disclosure.

1. An integrated machine for sterilizing and pressing, comprising:
a sterilization case, comprising an outer cylinder and an inner cylinder, wherein the inner cylinder is provided in the outer cylinder, a material mold is installed in the inner cylinder, and the material mold is provided with a plurality of material placing holes; and
pressing devices, wherein each of the pressing devices comprises a driving member and a pressing plate, and the driving member drives the pressing plate to move,
wherein the material mold is divided into X*Y pressing areas, an area of each of the pressing areas is equal to an area of the pressing plate, number of the pressing devices is X*Y, each of the pressing devices corresponds to each of the pressing areas, and a plurality of the material placing holes are distributed on the X*Y pressing areas.

2. The integrated machine for sterilizing and pressing according to claim 1, wherein a fixing frame is provided inside the inner cylinder, N layers of the material mold and N layers of the pressing devices are provided on the fixing frame along a height direction of the inner cylinder, and each of the layers of the material mold corresponds to each of the layers of the pressing devices.

3. The integrated machine for sterilizing and pressing according to claim 1, wherein the pressing plate is connected with a guiding structure, and a guiding direction of the guiding structure is parallel to a direction in which the driving member drives the pressing plate to move.

4. The integrated machine for sterilizing and pressing according to claim 3, wherein the driving member comprises a servo electric cylinder, the servo electric cylinder is provided with a telescopic rod, and the telescopic rod is fixedly connected to the pressing plate so as to drive the pressing plate to reciprocate with the telescopic rod.

5. The integrated machine for sterilizing and pressing according to claim 4, wherein the guiding structure comprises guiding rods and a guiding plate, an avoidance hole is provided at a central part of the guiding plate, the telescopic rod passes through the avoidance hole and is fixedly connected to the pressing plate, the guiding plate is provided with a plurality of guiding holes along a circumferential direction of the servo electric cylinder, an end of each of the guiding rods passes through or is inserted into the corresponding guiding hole and is slidable relative to the corresponding guiding hole, another end of each of the guiding rods is fixedly connected to the pressing plate, and both a number and a position of the guiding rods correspond to those of the guiding holes.

6. The integrated machine for sterilizing and pressing according to claim 1, wherein the material mold is provided with a bending-resistant reinforcing structure.

7. The integrated machine for sterilizing and pressing according to claim 6, wherein the bending-resistant reinforcing structure is a stress sandwich layer, the material mold comprises an upper structural plate and a lower structural plate, and the upper structural plate, the stress sandwich layer, and the lower structural plate are fixedly connected in sequence.

8. The integrated machine for sterilizing and pressing according to claim 7, wherein the stress sandwich layer is divided into two stress areas from a center of the stress sandwich layer to edges of the stress sandwich layer, one of the two stress areas at a central part of the stress sandwich layer is a first stress area, the other of the two stress areas at an edge part of the stress sandwich layer is a second stress area, and stress on the first stress area is greater than stress on the second stress area.

9. An assembly line for sterilizing and pressing a combination lid, comprising an integrated machine for sterilizing and pressing, configured to sterilize and press the combination lid, the integrated machine for sterilizing and pressing comprising:
a sterilization case, comprising an outer cylinder and an inner cylinder, wherein the inner cylinder is provided in the outer cylinder, a material mold is installed in the inner cylinder, and the material mold is provided with a plurality of material placing holes; and
pressing devices, wherein each of the pressing devices comprises a driving member and a pressing plate, and the driving member drives the pressing plate to move,
wherein the material mold is divided into X*Y pressing areas, an area of each of the pressing areas is equal to an area of the pressing plate, number of the pressing devices is X*Y, each of the pressing devices corresponds to each of the pressing areas, and a plurality of the material placing holes are distributed on the X*Y pressing areas; and
the combination lid comprises at least two components, two of the at least two components are correspondingly placed in one of the material placing holes, and the pressing devices press the two components together and then an enclosed cavity is formed between the two components.

10. The assembly line for sterilizing and pressing the combination lid according to claim 9, wherein a fixing frame is provided inside the inner cylinder, N layers of the material mold and N layers of the pressing devices are provided on the fixing frame along a height direction of the inner cylinder, and each of the layers of the material mold corresponds to each of the layers of the pressing devices.

11. The assembly line for sterilizing and pressing the combination lid according to claim 9, wherein the pressing plate is connected with a guiding structure, and a guiding direction of the guiding structure is parallel to a direction in which the driving member drives the pressing plate to move.

12. The assembly line for sterilizing and pressing the combination lid according to claim 11, wherein the driving member comprises a servo electric cylinder, the servo electric cylinder is provided with a telescopic rod, and the telescopic rod is fixedly connected to the pressing plate so as to drive the pressing plate to reciprocate with the telescopic rod.

13. The assembly line for sterilizing and pressing the combination lid according to claim 12, wherein the guiding structure comprises guiding rods and a guiding plate, an avoidance hole is provided at a central part of the guiding plate, the telescopic rod passes through the avoidance hole and is fixedly connected to the pressing plate, the guiding plate is provided with a plurality of guiding holes along a circumferential direction of the servo electric cylinder, an end of each of the guiding rods passes through or is inserted into the corresponding guiding hole and is slidable relative to the corresponding guiding hole, another end of each of the guiding rods is fixedly connected to the pressing plate, and both a number and a position of the guiding rods correspond to those of the guiding holes.

14. The assembly line for sterilizing and pressing the combination lid according to claim 9, wherein the material mold is provided with a bending-resistant reinforcing structure.

15. The assembly line for sterilizing and pressing the combination lid according to claim 14, wherein the bending-resistant reinforcing structure is a stress sandwich layer, the material mold comprises an upper structural plate and a lower structural plate, and the upper structural plate, the stress sandwich layer, and the lower structural plate are fixedly connected in sequence.

16. The assembly line for sterilizing and pressing the combination lid according to claim 15, wherein the stress sandwich layer is divided into two stress areas from a center of the stress sandwich layer to edges of the stress sandwich layer, one of the two stress areas at a central part of the stress sandwich layer is a first stress area, the other of the two stress areas at an edge part of the stress sandwich layer is a second stress area, and stress on the first stress area is greater than stress on the second stress area.

17. A method for sterilizing and pressing a combination lid, using an integrated machine for sterilizing and pressing, the integrated machine for sterilizing and pressing comprising:
a sterilization case, comprising an outer cylinder and an inner cylinder, wherein the inner cylinder is provided in the outer cylinder, a material mold is installed in the inner cylinder, and the material mold is provided with a plurality of material placing holes; and
pressing devices, wherein each of the pressing devices comprises a driving member and a pressing plate, and the driving member drives the pressing plate to move,
wherein the material mold is divided into X*Y pressing areas, an area of each of the pressing areas is equal to an area of the pressing plate, number of the pressing devices is X*Y, each of the pressing devices corresponds to each of the pressing areas, and a plurality of the material placing holes are distributed on the X*Y pressing areas;
the method specifically comprising following steps:
S1, stacking two components composing a combination lid in each of the material placing holes;
S2, placing the material mold into the integrated machine for sterilizing and pressing and closing a case door of the integrated machine for sterilizing and pressing;
S3, vacuuming the integrated machine for sterilizing and pressing to a vacuum state and heating up inside the integrated machine for sterilizing and pressing;
S4, spraying sterilizing gas into a sterilizing system inside the integrated machine for sterilizing and pressing, then setting pressure inside the integrated machine for sterilizing and pressing to a range of 0.1 MPa to 0.3 MPa and temperature inside the integrated machine for sterilizing and pressing to 115° C. to 121° C., and continuously spraying sterilizing gas for 15 minutes to 30 minutes to perform sterilization;
S5, vacuuming the integrated machine for sterilizing and pressing to the vacuum state again;
S6, drying inside the integrated machine for sterilizing and pressing to obtain a sterilized combination lid;
S7, pressing down X*Y pressing devices one by one until the last pressing device completes the pressing action, so as to complete pressing of the combination lid; and
S8, adjusting air pressure inside the integrated machine for sterilizing and pressing and opening a case door of the integrated machine for sterilizing and pressing, so as to complete production of the combination lid.

18. The assembly line for sterilizing and pressing the combination lid according to claim 17, wherein a fixing frame is provided inside the inner cylinder, N layers of the material mold and N layers of the pressing devices are provided on the fixing frame along a height direction of the inner cylinder, and each of the layers of the material mold corresponds to each of the layers of the pressing devices.

19. The method for sterilizing and pressing the combination lid according to claim 17, wherein the pressing plate is connected with a guiding structure, and a guiding direction of the guiding structure is parallel to a direction in which the driving member drives the pressing plate to move.

20. The method for sterilizing and pressing the combination lid according to claim 19, wherein the driving member comprises a servo electric cylinder, the servo electric cylinder is provided with a telescopic rod, and the telescopic rod is fixedly connected to the pressing plate so as to drive the pressing plate to reciprocate with the telescopic rod.
